(19) United States
(12) Patent Application Publication
(10) Pub. No.: US 2008/0014164 A1
(43) Pub. Date: Jan. 17, 2008

(54) MASCARA CONTAINING WAX AND FILLER

(75) Inventor: Isabelle Jacquier, Chevilly Larue (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/765,525

(22) Filed: Jun. 20, 2007

Related U.S. Application Data
(60) Provisional application No. 60/816,615, filed on Jun. 27, 2006.

(30) Foreign Application Priority Data
Jun. 20, 2006 (FR) 06 52545

(51) Int. Cl. A61K 8/92; A61Q 1/10

(52) U.S. Cl. 424/70.7

(57) ABSTRACT

Composition useful for coating keratinous fibres containing an aqueous phase, at least one filler, at least one film-forming polymer in the form of solid particles dispersed in the aqueous phase and at least one wax such that the total content of wax(es) represents at least 21% by weight with respect to the total weight of the composition, the composition exhibiting a viscosity at 25° C. of less than or equal to 13.5 Pa·s.

### REFERENCE TO RELATED CASES

This application claims priority to U.S. provisional application 60/816,615 filed Jun. 27, 2006, and to French patent application 0652545 filed Jun. 20, 2006, both incorporated herein by reference.

### FIELD OF THE INVENTION

The present invention relates to a composition useful especially for coating keratinous fibres, such as eyelashes, eyebrows or hair. It is provided in particular in the form of a mascara or of a product for the eyebrows. More especially, the invention relates to mascara.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The term “mascara” is understood to mean a composition intended to be applied to keratinous fibres: it can be a composition for making up the keratinous fibres, a base for making up keratinous fibres, a composition to be applied to mascara, also referred to as top coat, or else a composition for the cosmetic treatment of keratinous fibres. The mascara is more particularly intended for keratinous fibres of human beings but also for false eyelashes.

Generally, the compositions for making up keratinous fibres are composed of at least one wax or a mixture of waxes dispersed in an aqueous phase.

It is in particular through the amount of wax, which makes it possible to structure the composition, that the specific applicational features desired for the compositions, such as, for example, their fluidity or consistency, their covering power and/or their curving power, and their thickening power (also known as loading or making up power), are adjusted. As this type of composition sometimes exhibits insufficient hold on the eyelashes, it is known to use, in these compositions, film-forming polymers in the form of dispersions of solid particles in an aqueous phase (or latex). However, the use of latexes can result in a thickening of the composition and in a feeling of tackiness on application, and does not make it possible to obtain a smooth and homogeneous deposited layer on the eyelashes.

It is therefore difficult to obtain a composition for making up keratinous fibres comprising a high content of solids, and thus a satisfactory volumizing effect, while exhibiting easy and homogeneous application and good hold on keratinous fibres.

### SUMMARY OF THE INVENTION

One subject-matter of the invention is a composition useful especially for coating keratinous fibres comprising an aqueous phase, at least one filler, where the filler represents at least 0.1% by weight with respect to the total weight of the composition, at least one film-forming polymer in the form of solid particles dispersed in the aqueous phase and at least one wax such that the total content of wax(es) represents at least 21% by weight with respect to the total weight of the composition, the composition exhibiting a viscosity at 25° C. of less than or equal to 13.5 Pa·s.

A preferred composition according to the invention comprises a cosmetically acceptable medium, that is to say a medium which is non-toxic and capable of being applied to keratinous substances of human beings and with a pleasant appearance, pleasant smell and pleasant feel.

The present invention is also targeted at a process for caring for or making up keratinous fibres, wherein a composition in accordance with the invention is applied to the fibres.

It additionally relates to the use of a composition in accordance with the invention for obtaining a loading make-up for keratinous fibres, a smooth and homogeneous deposited layer and/or good hold on the keratinous fibres.

Within the meaning of the present invention, the term “loading” is intended to describe the notion of a thick and volumizing make-up for keratinous fibres, in particular eyelashes.

Preferably, the composition according to the invention is a leave-in composition.

Viscosity

The viscosity of the invention composition is measured at 25° C. using a Rheomat 180 (LAMY) equipped with an MS-R1, MS-R2, MS-R3, MS-R4 or MS-R5 spindle chosen according to the consistency of the composition which rotates at a rotational speed of 200 revolutions/min. The measurement is taken after rotating for 10 min. The viscosity measurements are carried out at most 1 week after manufacture.

The composition according to the invention preferably exhibits a viscosity of less than or equal to 13.5 Pa·s, ranging, for example, from 8 to 13.5 Pa·s, preferably from 9 to 13 Pa·s, better still from 9 to 12 Pa·s.

Wax(es)

Waxes useful in the context of the present invention include lipophilic compounds which are solid at ambient temperature (25° C.), which are or are not deformable, which exhibit a reversible solid/liquid change in state and which have a melting point of greater than or equal to 30° C. which can range up to 100° C. and in particular up to 90° C.

On bringing preferred waxes to the liquid state (melting), it is possible to render it miscible with oils and to form a microscopically homogeneous mixture but, on bringing the temperature of the mixture back to ambient temperature, recrystallization of the wax in the oils of the mixture is obtained. In particular, preferred waxes can exhibit a melting point of greater than or equal to 45° C. and in particular of greater than or equal to 55° C.

Within the meaning of the invention, the melting point corresponds to the temperature of the most endothermic peak observed by thermal analysis (DSC) as described in the standard ISO 11357-3; 1999. The melting point of the wax can be measured using a differential scanning calorimeter (DSC), for example the calorimeter sold under the name “MDSC 2920” by TA Instruments. The measurement protocol is as follows:

A 5 mg sample of wax placed in a crucible is subjected to a first rise in temperature ranging for example from −20° C. to 100° C. at a heating rate of 10° C./minute, is then cooled from 100° C. to −20° C. at a cooling rate of 10° C./minute and, finally, is subjected to a second rise in temperature ranging for example from −20° C. to 100° C. at a heating rate of 5° C./minute. During the second rise in temperature, the variation in the difference in power absorbed by the empty crucible and by the crucible comprising the sample of wax is measured as a function of the temperature. The melting point of the compound is the value of the temperature corresponding to the tip of the peak of the curve representing the variation in the difference in power absorbed as a function of the temperature.

Preferred waxes capable of being used in the compositions according to the invention include those chosen from waxes of animal, vegetable, mineral or synthetic origin, and their mixtures, which are solid at ambient temperature. Preferred waxes which can be used in the compositions according to the invention generally exhibit a hardness ranging for example from 0.01 MPa to 15 MPa, in particular of greater than 0.05 MPa and in particular of greater than 0.1 MPa.

The hardness is determined by the measurement of the compressive force measured at 20° C. using a texture analyser sold under the name TA-XT2 by Rheo, equipped with a stainless steel cylinder with a diameter of 2 mm which is displaced at the measuring rate of 0.1 mm/s and which penetrates the wax to a penetration depth of 0.3 mm.

The measurement protocol is as follows:

The wax is melted at a temperature equal to the melting point of the wax +10° C. The molten wax is cast in a receptacle with a diameter of 25 mm and a depth of 20 mm. The wax is recrystallized at ambient temperature (25° C.) for 24 hours, so that the surface of the wax is flat and smooth, and then the wax is stored at 20° C. for at least one hour before measuring the hardness or the tack.

The rotor of the texture analyser is displaced at a rate of 0.1 mm/s and then penetrates the wax to a penetration depth of 0.3 mm. When the rotor has penetrated the wax to the depth of 0.3 mm, the rotor is held stationary for 1 second (corresponding to the relaxation time) and is then withdrawn at the rate of 0.5 mm/s.

The value of the hardness is the maximum compressive force measured divided by the surface area of the cylinder of the texture analyser in contact with the wax.

Particularly useful waxes include: hydrocarbon waxes, such as beeswax, lanolin wax and Chinese insect waxes; rice bran wax, carnauba wax, candelilla wax, ouricury wax, alfa wax, berry wax, shellac wax, Japan wax and sumac wax; montan wax, orange and lemon waxes, microcrystalline waxes, paraffin waxes and ozokerite; polyethylene waxes, the waxes obtained by the Fischer-Tropsch synthesis and waxy copolymers, and their esters.

Mention may also be made of waxes obtained by catalytic hydrogenation of animal or vegetable oils having linear or branched C₈-C₃₂ fatty chains. Mention may in particular be made, among these, of isomerized jojoba oil, such as the transisomerized partially hydrogenated jojoba oil manufactured or sold by Desert Whale under the commercial reference Iso-Jojoba-50®, hydrogenated sunflower oil, hydrogenated castor oil, hydrogenated coconut oil, hydrogenated lanolin oil and di(1,1,1-trimethylolpropane)tetrastearate, sold under the name of Hest 2T-4S® by Heterene.

Mention may also be made of silicone waxes or fluorinated waxes. Use may also be made of the waxes obtained by hydrogenation of castor oil esterified with cetyl alcohol which are sold under the names of Phytowax Castor 16L64® and 22L73® by Sophim. Such waxes are disclosed in Application FR-A-2 792 190.

According to a preferred embodiment, the compositions according to the invention comprise at least one wax referred to as a “tacky wax”, that is to say having a tack of greater than or equal to 0.1 N.s and a hardness of less than or equal to 3.5 MPa. The tacky wax used can have in particular a tack ranging for example from 0.1 N.s to 10 N.s, in particular ranging for example from 0.1 N.s to 5 N.s, preferably ranging for example from 0.2 to 5 N.s and better still ranging for example from 0.3 to 2 N.s.

The tack of the wax is determined by the measurement of the change in the force (compressive force) as a function of the time at 20° C. according to the protocol indicated above for the hardness.

During the relaxation time of 1 s, the force (compressive force) strongly decreases until it becomes zero and then, during the withdrawal of the rotor, the force (stretching force) becomes negative to subsequently again increase towards the value 0. The tack corresponds to the integral of the curve of the force as a function of the time for the part of the curve corresponding to the negative values of the force. The value of the tack is expressed in N.s.

A preferred tacky wax which can be used generally has a hardness of less than or equal to 3.5 MPa, in particular ranging for example from 0.01 MPa to 3.5 MPa, especially ranging for example from 0.05 MPa to 3 MPa.

Use may be made, as tacky wax, of a C₂₀-C₄₀ alkyl (hydroxystearyloxy)stearate (the alkyl group comprising from 20 to 40 carbon atoms), alone or as a mixture.

Such a wax is sold in particular under the names “Kester Wax K 82 P®∞, “Hydroxypolyester K 82 P®” and “Kester Wax K 80 P®” by Koster Keunen.

In the present invention, use may be made of waxes provided in the form of small particles having a size, expressed as volume-average “effective” diameter D[4,3], of the order of 0.5 to 30 micrometres, in particular of 1 to 20 micrometres and more particularly of 5 to 10 micrometres, subsequently denoted by the expression “microwaxes”. The sizes of the particles can be measured by various techniques. Mention may in particular be made of light scattering techniques (dynamic and static), Coulter counter methods, measurements by rate of sedimentation (related to the size via Stokes' law) and microscopy. These techniques make it possible to measure a particle diameter and, for some of them, a particle size distribution.

Preferably, the sizes and size distributions of the particles of the compositions according to the invention are measured by static light scattering using a commercial particle sizer of MasterSizer 2000 type from Malvern. The data are processed on the basis of the Mie scattering theory. This theory, exact for isotropic particles, makes it possible to determine, in the case of nonspherical particles, an “effective” particle diameter. This theory is described in particular in the work by Van de Hulst, H. C., “Light Scattering by Small Particles”, Chapters 9 and 10, Wiley, New York, 1957.

The composition is characterized by its volume-average “effective” diameter D[4,3], defined in the following way:

$$D [ 4 , 3 ] = \frac{{\sum }_{i} {V}_{i} \cdot {d}_{i}}{{\sum }_{i} {V}_{i}}$$

where Vᵢ represents the volume of the particles with an effective diameter dᵢ. This parameter is described in particular in the technical documentation of the particle sizer.

The measurements are carried out at 25° C. on a diluted dispersion of particles obtained from the composition in the following way: 1) dilution with water by a factor of 100, 2) homogenization of the solution, 3) standing the solution for 18 hours, 4) recovery of the off-white homogeneous supernatant.

The “effective” diameter is obtained by taking a refractive index of 1.33 for the water and a mean refractive index of 1.42 for the particles.

Useful microwaxes which can be used in the compositions according to the invention include carnauba microwaxes, such as that sold under the name of MicroCare 350® by Micro Powders, synthetic wax microwaxes, such as that sold under the name of MicroEase 114S® by Micro Powders, microwaxes composed of a mixture of carnauba wax and of polyethylene wax, such as those sold under the names of MicroCare 300® and 310® by Micro Powders, microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, and polytetrafluoroethylene microwaxes, such as those sold under the names of Microslip 519® and 519 L® by Micro Powders.

The composition according to the invention exhibits a content of waxes of greater than or equal to 21% by weight with respect to the total weight of the composition, preferably greater than or equal to 22% by weight, with a preferred upper limit of 40% by weight, better still 35% by weight, preferably 30% by weight, including 23, 24, 25, 26, 27, 28, 29, 30, 31, 32, 33, 34, 35, 36, 37, 38, 39 and 40% as well as all values and subranges between stated values. Upper limits of 45, 50, 55, 60% by weight are possible, but not preferred.

Fillers

The fillers can be inorganic or organic and lamellar or spherical and preferably represent at least 0.1% by weight with respect to the total weight of the composition, for example from 0.1 to 25% by weight, preferably at least 0.5% by weight, for example from 0.5 to 25% by weight, better, at least 1% by weight, in particular from 1 to 20% by weight.

Generally, the fillers used according to the invention are preferably colourless or white, namely non pigmentary, that is to say that they are not used to confer a specific colour or tint on the composition according to the invention, even if their use may intrinsically lead to such a result. They are thus, as such, preferably distinct from pearlescent agents, organic pigments, such as, for example, carbon black, pigments of D & C type and lakes, based on cochineal carmine, of barium, strontium, calcium or aluminium, and inorganic pigments, such as, for example, titanium dioxide, zirconium or cerium oxides, and also oxides of iron (black, yellow or red) or chromium, manganese violet, ultramarine blue, chromium hydrate and ferric blue, which, themselves, are used to provide a tinting and colouring effect to compositions incorporating them. Such compounds are not covered, within the meaning of the invention, by the definition of nonpigmentary fillers.

The fillers according to the invention may or may not be coated at the surface, in particular surface treated, by silicones, amino acids, fluorinated derivatives or any other substance promoting the dispersion and the compatibility of the filler in the composition.

According to one embodiment, the composition according to the invention comprises at least one inorganic filler and at least one organic filler.

Preferred nonpigmentary inorganic fillers which can be used in the compositions according to the invention include: talc, mica, silica, kaolin, starch, hydroxyapatite, boron nitride, hollow silica microspheres (silica beads from Maprecos), glass or ceramic microcapsules, and their mixtures.

Preferred organic fillers useful herein include powders formed of polyamide (Nylon®, Orgasol from Atochem), of poly-β-alanine or of polyethylene, lauroyllysine, starch, powders formed of tetrafluoroethylene polymers (such as Teflon), expanded polymer hollow microspheres, such as those of poly(vinylidene chloride)/acrylonitrile, for example those sold under the name of Expancel® by Nobel Industrie, acrylic powders, such as those sold under the name Polytrap® by Dow Corning, acrylate copolymers, poly(methyl methacrylate) (PMMA), 12-hydroxystearic acid oligomer stearate, silicone resin microbeads (Tospearis from Toshiba, for example), precipitated calcium carbonate, magnesium carbonate, basic magnesium carbonate, metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, heat-expandable particles, such as nonexpanded microspheres of vinylidene chloride/acrylonitrile/methyl methacrylate copolymer or of copolymer of homopolymer of acrylonitrile, such as, for example, those sold respectively under the references Expancel® 820 DU 40 and Expancel® 007WU by Akzo Nobel, and their mixtures.

According to one embodiment, the composition according to the invention comprises at least one inorganic filler, preferably chosen from silicas, preferably silica microbeads or microspheres, and at least one organic filler, preferably chosen from polyethylene powders. According to a specific embodiment, the inorganic filler is present in a proportion of 0.1 to 10% by weight, in particular of 0.5 to 5% by weight, with respect to the total weight of the composition, and the organic filler is present in a proportion of 0.05 to 5% by weight, in particular of 0.1 to 2% by weight, with respect to the total weight of the composition.

Aqueous Phase

The composition according to the invention comprises an aqueous medium, constituting an aqueous phase, which can form the continuous phase of the composition.

The aqueous phase of the composition according to the invention is advantageously a continuous aqueous phase. The term “composition comprising a continuous aqueous phase” is understood to mean that the composition exhibits a conductivity, measured at 25° C., of greater than 23 μS/cm (microSiemens/cm), the conductivity being measured, for example, using an MPC227 conductivity meter from Mettler Toledo and an Inlab730 conductivity measurement cell. The measurement cell is immersed in the composition, so as to remove the air bubbles capable of being formed between the 2 electrodes of the cell. The conductivity is read as soon as the value of the conductivity meter has stabilized. A mean is taken over at least 3 successive measurements.

The aqueous phase can be composed completely or essentially of water; it can also comprise a mixture of water and of water-miscible solvent(s) (miscibility in water of greater than 50% by weight at 25° C.), such as low monoalcohols having from 1 to 5 carbon atoms, such as ethanol or isopropanol, glycols having from 2 to 8 carbon atoms, such as propylene glycol, ethylene glycol, 1,3-butylene glycol or dipropylene glycol, C₃-C₄ ketones, C₂-C₄ aldehydes, etc., and their mixtures.

The aqueous phase (water and optionally the water-miscible solvent) can be present in a content ranging for example from 1% to 95% by weight, with respect to the total weight of the composition, preferably ranging for example from 3% to 80% by weight and preferentially ranging for example from 5% to 60% by weight.

Emulsifying System

The compositions according to the invention can optionally comprise emulsifying surface-active agents present in particular in a proportion ranging for example from 0.1% to 20% and better still from 0.3% to 15% by weight, with respect to the total weight of the composition.

According to the invention, use is generally made of an emulsifier appropriately chosen in order to obtain an oil-in-water emulsion. Use may in particular be made of an emulsifier having, at 25° C., an HLB (hydrophilic-lipophilic balance) balance within the meaning of Griffin of greater than or equal to 8. The HLB value according to Griffin is defined in J. Soc. Cosm. Chem., 1954 (volume 5), pages 249-256. These surface-active agents can be chosen from nonionic, anionic, cationic or amphoteric surface-active agents or also surface-active emulsifiers. Reference may be made to the document “Encyclopedia of Chemical Technology, Kirk-Othmer”, volume 22, pp. 333-432, 3rd edition, 1979, Wiley, for the definition of the properties and functions (emulsifying) of surfactants, in particular pp. 347-377 of this reference for the anionic, amphoteric and nonionic surfactants.

The surfactants preferentially used in the composition according to the invention include:

a) nonionic surface-active agents with an HLB of greater than or equal to 8 to 25° C., used alone or as a mixture; mention may in particular be made of:
oxyethylenated and/or oxypropylenated ethers (which can comprise from 1 to 150 oxyethylene and/or oxypropylene groups) of glycerol;
oxyethylenated and/or oxypropylenated ethers (which can comprise from 1 to 150 oxyethylene and/or oxypropylene groups) of fatty alcohols (in particular of C₈-C₂₄ and preferably C₁₂-C₁₈ alcohols), such as the oxyethylenated ether of stearyl alcohol comprising 20 oxyethylene groups (CTFA name “Steareth-20”), such as Brij 78, sold by Uniquema, the oxyethylenated ether of cetearyl alcohol comprising 30 oxyethylene groups (CTFA name “Ceteareth-30”) and the oxyethylenated ether of the mixture of C₁₂-C₁₅ fatty alcohols comprising 7 oxyethylene groups (CTFA name “C12-15 Pareth-7”) sold under the name Neodol 25-7® by Shell Chemicals,
esters of fatty acid (in particular of C₈-C₂₄ and preferably C₁₆-C₂₂ acid) and of polyethylene glycol (which can comprise from 1 to 150 ethylene glycol units), such as PEG-50 stearate and PEG-40 monostearate, sold under the name Myrj 52P® by ICI Uniquema,
esters of fatty acid (in particular of C₈-C₂₄ and preferably C₁₆-C₂₂ acid) and of the oxyethylenated and/or oxypropylenated glycerol ethers (which can comprise from 1 to 150 oxyethylene and/or oxypropylene groups), such as PEG-200 glyceryl monostearate, sold under the name Simulsol 220 TM® by Seppic; polyethoxylated glyceryl stearate comprising 30 ethylene oxide groups, such as the product Tagat S® sold by Goldschmidt, polyethoxylated glyceryl oleate comprising 30 ethylene oxide groups, such as the product Tagat O® sold by Goldschmidt, polyethoxylated glyceryl cocoate comprising 30 ethylene oxide groups, such as the product Varionic LI 13® sold by Sherex, polyethoxylated glyceryl isostearate comprising 30 ethylene oxide groups, such as the product Tagat L® sold by Goldschmidt and polyethoxylated glyceryl laurate comprising 30 ethylene oxide groups, such as the product Tagat I® from Goldschmidt,
esters of fatty acid (in particular of C₈-C₂₄ and preferably C₁₆-C₂₂ acid) and of the oxyethylenated and/or oxypropylenated sorbitol ethers (which can comprise from 1 to 150 oxyethylene and/or oxypropylene groups), such as polysorbate 60, sold under the name Tween 60® by Uniquema,
dimethicone copolyol, such as that sold under the name Q2-5220® by Dow Corning,
dimethicone copolyol benzoate (Finsolv SLB 101® and 201® from Fintex),
copolymers of propylene oxide and of ethylene oxide, also known as EO/PO polycondensates,
and their mixtures.

The EO/PO polycondensates include copolymers of polyethylene glycol and polypropylene glycol blocks, such as, for example, polyethylene glycol/polypropylene glycol/polyethylene glycol triblock polycondensates. These triblock polycondensates have, for example, the following chemical structure:
H—(O—CH₂—CH₂)ₐ—(O—CH(CH₃)—CH₂)_{b}—(O—CH₂—CH₂)ₐ—OH,
in which formula a ranges from 2 to 120 and b ranges from 1 to 100.

The EO/PO polycondensate preferably has a weight-average molecular weight ranging for example from 1000 to 15 000 and better still ranging for example from 2000 to 13 000. Advantageously, the EO/PO polycondensate has a cloud point, at 10 g/l in distilled water, of greater than or equal to 20° C., preferably of greater than or equal to 60° C. The cloud point is measured according to the standard ISO 1065. Mention may be made, as EO/PO polycondensate which can be used according to the invention, of the polyethylene glycol/polypropylene glycol/polyethylene glycol triblock polycondensates sold under the Synperonic® names, such as Synperonic PE/L44® and Synperonic PE/F127®, by ICI,
b) nonionic surface-active agents with an HLB of less than 8 at 25° C., optionally in combination with one or more nonionic surface-active agents with an HLB of greater than 8 at 25° C., such as mentioned above, such as:
esters and ethers of monosaccharides, such as sucrose stearate, sucrose cocoate, sorbitan stearate and their mixtures, such as Arlatone 2121®, sold by ICI, or Span 65V, from Uniquema;
esters of fatty acids (in particular of C₈-C₂₄ and preferably C₁₆-C₂₂ acid) and of polyol, in particular of glycerol or of sorbitol, such as glyceryl stearate, such as the product sold under the name Tegin M® by Goldschmidt, glyceryl laurate, such as the product sold under the name Imwitor 312® by Hüls, polyglyceryl-2 stearate, sorbitan tristearate or glyceryl ricinoleate;
oxyethylenated and/or oxypropylenated ethers, such as the oxyethylenated ether of stearyl alcohol comprising 2 oxyethylene groups (CTFA name “Steareth-2”), such as Brij 72, sold by Uniquema;
the cyclomethicone/dimethicone copolyol mixture sold under the name Q2-3225C® by Dow Corning,
c) anionic surfactants, such as:
salts of polyoxyethylenated fatty acids, in particular those derived from amines or the alkali metal salts, and their mixtures;
phosphoric esters and their salts, such as “DEA oleth-10 phosphate” (Crodafos N 10N from Croda) or monopotassium monocetyl phosphate (Amphisol K from Givaudan or Arlatone MAP 160K from Uniquema);
sulphosuccinates, such as “Disodium PEG-5 citrate lauryl sulphosuccinate” and “Disodium ricinoleamido MEA sulphosuccinate”;
alkyl ether sulphates, such as sodium lauryl ether sulphate;
isethionates;
acylglutamates, such as “Disodium hydrogenated tallow glutamate” (Amisoft HS-21 R®, sold by Ajinomoto), and their mixtures.

Mention may in particular be made, by way of representation of cationic surfactants, of:
alkyl imidazolidiniums, such as isostearyl ethylimidonium ethosulphate,
ammonium salts, such as N,N,N-trimethyl-1-docosanaminium chloride (behentrimonium chloride).

The compositions according to the invention can also comprise one or more amphoteric surfactants, such as N-acylamino acids, for example N-acylaminoacetates and disodium cocoamphodiacetate, and amine oxides, such as stearamine oxide, or also silicone surfactants, such as dimethicone copolyol phosphates, such as that sold under the name Pecosil PS 100® by Phoenix Chemical.

Water-Soluble Gelling Agent

The composition according to the invention can comprise a water-soluble gelling agent.

The water-soluble gelling agents which can be used in the compositions according to the invention include those chosen from:
homo- or copolymers of acrylic acid or methacrylic acid or their salts and their esters and in particular the products sold under the names Versicol F® or Versicol K® by Allied Colloid, Ultrahold 8® by Ciba-Geigy, poly(acrylic acid)s of Synthalen K type,
copolymers of acrylic acid and of acrylamide, sold in the form of their sodium salts under the Reten® names by Hercules, poly(sodium methacrylate), sold under the name Darvan No. 7® by Vanderbilt, sodium salts of poly(hydroxycarboxylic acid)s, sold under the name Hydagen F® by Henkel,
copolymers of poly(acrylic acid)s and of alkyl acrylates of Pemulen type,
AMPS (poly(acrylamidomethylpropanesulphonic acid) partially neutralized with aqueous ammonia and highly crosslinked), sold by Clariant,
AMPS/acrylamide copolymers of Sepigel® or Simugel® type, sold by Seppic, and
copolymers of AMPS and of alkyl methacrylates which are polyoxyethylenated (crosslinked or noncrosslinked),
proteins, such as proteins of plant origin, such as wheat or soya proteins; proteins of animal origin, such as keratins, for example keratin hydrolysates and sulphonic keratins;
cellulose polymers, such as hydroxyethylcellulose, hydroxypropylcellulose, methylcellulose, ethylhydroxyethylcellulose, carboxymethylcellulose and quaternized cellulose derivatives;
acrylic polymers or copolymers, such as polyacrylates or polymethacrylates;
vinyl polymers, such as polyvinylpyrrolidones, copolymers of methyl vinyl ether and of malic anhydride, the copolymer of vinyl acetate and of crotonic acid, copolymers of vinylpyrrolidone and of vinyl acetate, copolymers of vinylpyrrolidone and of caprolactam, or poly(vinyl alcohol);
optionally modified polymers of natural origin, such as:
gums arabic, guar gum, xanthan derivatives or karaya gum;
alginates and carrageenans;
glycoaminoglycans, hyaluronic acid and its derivatives;
shellac resin, gum sandarac, dammars, elemis or copals;
deoxyribonucleic acid;
mucopolysaccharides, such as chondroitin sulphates,
and their mixtures.

Some of these water-soluble gelling agents can also act as film-forming polymers.

The water-soluble gelling polymer can be present in the composition according to the invention in a content of dry matter ranging for example from 0.01% to 60% by weight, preferably from 0.5% to 40% by weight, better still from 1% to 30% by weight, indeed even from 5% to 20% by weight, with respect to the total weight of the composition.

Film-Forming Polymer

The composition according to the invention can advantageously comprise at least one film-forming polymer.

The film-forming polymer can be present in the composition according to the invention in a content of dry matter (or active materials) ranging for example from 0.1% to 30% by weight, with respect to the total weight of the composition, preferably from 0.5% to 20% by weight and better still from 1% to 15% by weight. Preferably, the composition comprises at least 2% by weight of dry matter of film-forming polymer, preferably at least 3% by weight.

In the present invention, the term “film-forming polymer” is understood to mean a polymer capable of forming, alone or in the presence of an additional agent which is able to form a film, a macroscopically continuous film which adheres to keratinous fibres, preferably a cohesive film, better still a film possessing a cohesion and mechanical properties such that the film may be able to be isolated and to be handled in isolation, for example when the film is produced by casting over a non-stick surface, such as a Teflon or silicone surface. Included among the film-forming polymers which can be used in the composition of the present invention are: synthetic polymers of radical type or of polycondensate type, polymers of natural origin, and their mixtures.

The term “radical film-forming polymer” is understood to mean a polymer obtained by polymerization of monomers possessing unsaturation, in particular ethylenic unsaturation, each monomer being capable of homopolymerizing (unlike polycondensates).

The film-forming polymers of radical type can in particular be vinyl polymers or copolymers, in particular acrylic polymers.

The film-forming vinyl polymers can result from the polymerization of monomers possessing ethylenic unsaturation having at least one acid group and/or of the esters of these acidic monomers and/or of the amides of these acidic monomers.

Use may be made, as monomer carrying an acid group, of, for example, unsaturated α,β-ethylenic carboxylic acids, such as acrylic acid, methacrylic acid, crotonic acid, maleic acid or itaconic acid. Use is preferably made of (meth)acrylic acid and crotonic acid and more preferentially of (meth)acrylic acid.

The esters of acidic monomers are advantageously chosen from esters of (meth)acrylic acid (also known as (meth)acrylates), in particular alkyl(meth)acrylates, especially C₁-C₃₀ alkyl(meth)acrylates, preferably C₁-C₂₀ alkyl(meth)acrylates, aryl(meth)acrylates, in particular C₆-C₁₀ aryl(meth)acrylates, hydroxyalkyl(meth)acrylates, in particular C₂-C₆ hydroxyalkyl(meth)acrylates.

Mention may be made, among alkyl(meth)acrylates, of for example methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl(meth)acrylates, of for example hydroxyethyl acrylate, 2-hydroxypropyl acrylate, hydroxyethyl methacrylate or 2-hydroxypropyl methacrylate.

Mention may be made, among aryl(meth)acrylates, of for example benzyl acrylate and phenyl acrylate.

Esters of (meth)acrylic acid which are particularly preferred are alkyl(meth)acrylates. According to the present invention, the alkyl group of the esters can be either fluorinated or perfluorinated, that is to say that a portion or all of the hydrogen atoms of the alkyl group are substituted by fluorine atoms.

Mention may be made, as amides of the acidic monomers, for example, of (meth)acrylamides, in particular N-alkyl(meth)acrylamides, especially N-(C₂-C₁₂ alkyl)(meth)acrylamides. Mention may be made, among N-alkyl(meth)acrylamides, of N-ethylacrylamide, N-(t-butyl)acrylamide, N-(t-octyl)acrylamide and N-undecylacrylamide.

The film-forming vinyl polymers can also result from the homopolymerization or from the copolymerization of monomers chosen from vinyl esters and styrene monomers. In particular, these monomers can be polymerized with acidic monomers and/or their esters and/or their amides, such as those mentioned above.

Mention may be made, as examples of vinyl esters, of for example vinyl acetate, vinyl neodecanoate, vinyl pivalate, vinyl benzoate and vinyl t-butylbenzoate.

Mention may be made, as styrene monomers, of for example styrene and α-methylstyrene. Mention may be made, among film-forming polycondensates, of polyurethanes, polyesters, polyesteramides, polyamides, epoxy ester resins or polyureas.

The polyurethanes can be chosen from anionic, cationic, nonionic or amphoteric polyurethanes, polyurethane-acrylics, polyurethane-polyvinylpyrrolidones, polyester-polyurethanes, polyether-polyurethanes, polyureas, polyurea-polyurethanes, and their blends. The polyesters can be obtained in a known way by polycondensation of dicarboxylic acids with polyols, in particular diols. The dicarboxylic acid can be aliphatic, alicyclic or aromatic. Mention may be made, as examples of such acids, of oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers can be used alone or as a combination of at least two dicarboxylic acid monomers. The choice is preferentially made, among these monomers, of phthalic acid, isophthalic acid or terephthalic acid. The diol can be chosen from aliphatic, alicyclic or aromatic diols. Use is preferably made of a diol chosen from ethylene glycol, diethylene glycol, triethylene glycol, 1,3-propanediol, cyclohexanedi methanol or 1,4-butanediol. Use may be made, as other polyols, of glycerol, pentaerythritol, sorbitol or trimethylolpropane. The polyesteramides can be obtained in an analogous way to the polyesters, by polycondensation of diacids with diamines or aminoalcohols. Use may be made, as diamine, of ethylenediamine, hexamethylenediamine, meta-phenylenediamine or para-phenylenediamine. Use may be made, as aminoalcohol, of monoethanolamine. The polyester can additionally comprise at least one monomer carrying at least one —SO₃M group, with M representing a hydrogen atom, an NH₄⁺ ammonium ion or a metal ion, such as, for example, an Na⁺, Li⁺, K⁺, Mg²⁺, Ca²⁺, Cu²⁺, Fe²⁺ or Fe³⁺ ion. Use may in particular be made of a bifunctional aromatic monomer comprising such an —SO₃M group.

The aromatic nucleus of the bifunctional aromatic monomer additionally carrying an —SO₃M group as described above can be chosen, for example, from the benzene, naphthalene, anthracene, diphenyl, oxydiphenyl, sulphonyidiphenyl or methylenediphenyl nuclei. Mention may be made, as example of bifunctional aromatic monomer additionally carrying an —SO₃M group, of sulphoisophthalic acid, sulphoterephthalic acid, sulphophthalic acid or 4-sulphonaphthalene-2,7-dicarboxylic acid.

Preference is given to the use of copolymers based on isophthalate/sulphoisophthalate and more particularly to copolymers obtained by condensation of diethylene glycol, cyclohexanedimethanol, isophthalic acid and sulphoisophthalic acid.

The optionally modified polymers of natural origin can be chosen from shellac resin, gum sandarac, dammars, elemis, copals, cellulose polymers and their blends.

The film-forming polymer is advantageously present in the composition in the form of solid particles in dispersion in the aqueous phase, generally known under the name of latex or pseudolatex. The techniques for the preparation of these dispersions are well known to a person skilled in the art. Use may be made, as aqueous film-forming polymer dispersion, of for example acrylic dispersions, sold under the names Neocryl XK-90®, Neocryl A-1070®, Neocryl A-1090®, Neocryl BT-62®, Neocryl A-1079® and Neocryl A-523® by Avencia Neoresins, Dow Latex 432® by Dow Chemical, Daitosol 5000 AD® or Daitosol 5000 SJ® by Daito Kasey Kogyo; Syntran 5760® by Interpolymer, Allianz OPT by Röhm & Haas, aqueous dispersions of acrylic or styrene/acrylic polymers, sold under the trade name Joncryl® by Johnson Polymer, or aqueous dispersions of polyurethane, sold under the names Neorez R-981® and Neorez R-974® by Avecia-Neoresins, Avalure UR-405®, Avalure UR-410®, Avalure UR-425®, Avalure UR-450®, Sancure 875®, Sancure 861®, Sancure 878® and Sancure 2060® by Goodrich, Impranil 85® by Bayer, Aquamere H-1511® by Hydromer; sulphopolyesters, sold under the trade name Eastman AQ® by Eastman Chemical Products, vinyl dispersions, such as Mexomer PAM® from Chimex, and their blends.

According to an advantageous embodiment, the composition according to the invention comprises at least one film-forming acrylic polymer in the form of solid particles in dispersion in the aqueous phase, the polymer preferably resulting from the polymerization of at least one monomer possessing ethylenic unsaturation chosen from α,β-ethylenic carboxylic acids, their esters and their amides.

Use may be made, as α,β-ethylenic unsaturated carboxylic acid, of for example acrylic acid, methacrylic acid, crotonic acid, maleic acid or itaconic acid. Use is preferably made of (meth)acrylic acid and crotonic acid and more preferentially of (meth)acrylic acid.

The esters of these carboxylic acids can be chosen from for example esters of (meth)acrylic acid (also known as (meth)acrylates), in particular alkyl(meth)acrylates, especially C₁-C₃₀ alkyl(meth)acrylates, preferably C₁-C₂₀ alkyl(meth)acrylates, aryl(meth)acrylates, in particular C₆-C₁₀ aryl(meth)acrylates, or hydroxyalkyl(meth)acrylates, in particular C₂-C₆ hydroxyalkyl(meth)acrylates.

Mention may be made, among alkyl(meth)acrylates, of for example methyl methacrylate, ethyl methacrylate, butyl methacrylate, isobutyl methacrylate, 2-ethylhexyl methacrylate, lauryl methacrylate or cyclohexyl methacrylate.

Mention may be made, among hydroxyalkyl(meth)acrylates, of for example hydroxyethyl acrylate, 2-hydroxypropyl acrylate, hydroxyethyl methacrylate or 2-hydroxypropyl methacrylate.

Mention may be made, among aryl(meth)acrylates, of for example benzyl acrylate and phenyl acrylate.

It is possible, of course, to employ a mixture of these monomers.

The esters of (meth)acrylic acid which are particularly preferred are alkyl(meth)acrylates.

The alkyl group of the esters can optionally be either fluorinated or perfluorinated, that is to say that a portion or all of the hydrogen atoms of the alkyl group are substituted by fluorine atoms.

Mention may be made, as amides of the carboxylic acids, for example, of (meth)acrylamides and in particular N-alkyl(meth)acrylamides, especially N-(C₂-C₁₂ alkyl)(meth)acrylamides. Mention may be made, among N-alkyl(meth)acrylamides, of N-ethylacrylamide, N-(t-butyl)acrylamide, N-(t-octyl)acrylamide and N-undecylacrylamide.

The film-forming acrylic polymer which can be used according to the invention can comprise, in addition to the monomers mentioned above, at least one styrene monomer, such as styrene or α-methylstyrene.

Use may be made, as acrylic polymer, of those sold under the names “Syntran® 5190”, “Syntran® 5760” or “Syntran® 5009” by Interpolymer or “Dow Latex 424®” by Dow Chemical.

The composition according to the invention can also comprise an “additional” film-forming polymer which can be a water-soluble polymer dissolved in the aqueous phase of the composition.

The film-forming polymer is preferably present in the composition in the form of particles in dispersion in an aqueous phase or in a nonaqueous solvent phase. According to another alternative embodiment of the composition according to the invention, the additional film-forming polymer can be a polymer dissolved in a liquid fatty phase comprising oils or organic solvents, such as those described above (the film-forming polymer is then described as a fat-soluble polymer), or can be present in the composition in the form of particles in dispersion in a nonaqueous solvent phase. Preferably, the liquid fatty phase comprises a volatile oil, optionally as a mixture with a non-volatile oil, it being possible for the oils to be chosen from the oils mentioned above.

Mention may be made, as examples of fat-soluble polymer, of for example copolymers of vinyl ester (the vinyl group being directly connected to the oxygen atom of the ester group and the vinyl ester having a saturated, linear or branched, hydrocarbon radical of 1 to 19 carbon atoms bonded to the carbonyl of the ester group) and of at least one other monomer which can be a vinyl ester (other than the vinyl ester already present), an α-olefin (having from 8 to 28 carbon atoms), an alkyl vinyl ether (the alkyl group of which comprises from 2 to 18 carbon atoms) or an allyl or methallyl ester (having a saturated, linear or branched, hydrocarbon radical of 1 to 19 carbon atoms bonded to the carbonyl of the ester group).

These copolymers can be crosslinked using crosslinking agents which can be either of the vinyl type or of the allyl or methallyl type, such as tetraallyloxyethane, divinylbenzene, divinyl octanedioate, divinyl dodecanedioate and divinyl octadecanedioate.

Mention may be made, as examples of these copolymers, of for example the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% of divinylbenzene, vinyl acetate/octadecyl vinyl ether, crosslinked with 0.2% of tetraallyloxyethane, vinyl acetate/allyl stearate, crosslinked with 0.2% of divinylbenzene, vinyl acetate/1-octadecene, crosslinked with 0.2% of divinylbenzene, and allyl propionate/allyl stearate, crosslinked with 0.2% of divinylbenzene.

Mention may also be made, as fat-soluble film-forming polymers, of for example fat-soluble copolymers and in particular those resulting from the copolymerization of vinyl esters having from 9 to 22 carbon atoms or of alkyl acrylates or methacrylates, the alkyl radicals having from 10 to 20 carbon atoms. Such fat-soluble copolymers can for example be chosen from copolymers of poly(vinyl stearate), of poly(vinyl stearate) crosslinked using divinylbenzene, diallyl ether or diallyl phthalate, copolymers of poly(stearyl (meth)acrylate), of poly(vinyl laurate), of poly(lauryl(meth)acrylate), it being possible for these poly(meth)acrylates to be crosslinked using ethylene glycol dimethacrylate or tetraethylene glycol dimethacrylate.

The fat-soluble copolymers defined above are known and are disclosed in particular in Application FR-A-2 232 303; they can have a weight-average molecular weight ranging for example from 2000 to 500 000 and preferably from 4000 to 200 000.

Mention may also be made, as fat-soluble film-forming polymers which can be used in the invention, of for example polyalkylenes and in particular copolymers of C₂-C₂₀ alkenes, such as polybutene, alkylcelluloses with a saturated or unsaturated and linear or branched C₁ to C₈ alkyl radical, such as ethylcellulose and propylcellulose, copolymers of vinylpyrrolidone (VP) and in particular copolymers of vinylpyrrolidone and of C₂ to C₄₀ alkene and better still C₃ to C₂₀ alkene. Mention may be made, as examples of VP copolymer which can be used in the invention, of for example the VP/vinyl acetate, VP/ethyl methacrylate, VP/ethyl methacrylate/methacrylic acid, VP/eicosene, VP/hexadecene, VP/triacontene, VP/styrene or VP/acrylic acid/lauryl methacrylate copolymer or butylated polyvinylpyrrolidone (PVP).

Mention may also be made of silicone resins, generally soluble or swellable in silicone oils, which are crosslinked polyorganosiloxane polymers. The nomenclature of silicone resins is known under the name of “MDTQ”, the resin being described according to the various siloxane monomer units which it comprises, each of the letters “MDTQ” characterizing one type of unit.

Mention may be made, as examples of commercially available polymethylsilsesquioxane resins, of those which are sold:
by Wacker under the reference Resin MK, such as Belsil PMS MK;
by Shin-Etsu under the references KR-220L.

Mention may be made, as siloxysilicate resins, of trimethylsiloxysilicate (TMS) resins, such as those sold under the reference SR1000 by General Electric or under the reference TMS 803 by Wacker. Mention may also be made of trimethylsiloxysilicate resins sold in a solvent, such as cyclomethicone, sold under the names “KF-7312J” by Shin-Etsu or “DC 749” or “DC 593” by Dow Corning.

Mention may also be made of copolymers of silicone resins, such as those mentioned above with polydimethylsiloxanes, such as for example the pressure-sensitive adhesive copolymers sold by Dow Corning under the reference BIO-PSA and disclosed in the document U.S. Pat. No. 5,162,410 or the silicone copolymers resulting from the reaction of a silicone resin, such as those described above, and of a diorganosiloxane, such as are disclosed in the document WO 2004/073626.

According to one embodiment of the invention, the film-forming polymer is a film-forming linear block ethylenic polymer which preferably comprises at least one first block and at least one second block having different glass transition temperatures (Tg), the first and second blocks being connected to one another via an intermediate block comprising at least one constituent monomer of the first block and at least one constituent monomer of the second block.

Advantageously, the first and second blocks of the block polymer are incompatible with one another.

Such polymers are disclosed, for example, in the documents EP 1 411 069 or WO04/028488.

Mention may be made, as examples of nonaqueous dispersions of film-forming polymer, of acrylic dispersions in isododecane, such as Mexomer PAP® from Chimex, dispersions of particles of a grafted ethylenic polymer, preferably an acrylic polymer, in a liquid fatty phase, the ethylenic polymer advantageously being dispersed in the absence of additional stabilizer at the surface of the particles, such as disclosed in particular in the document WO 04/055081.

The composition according to the invention can comprise a plasticizing agent favourable to the formation of a film with the film-forming polymer. Such a plasticizing agent can be chosen from any compound known to a person skilled in the art as being capable of fulfilling the desired role.

Oils

The composition can comprise one or more oils. The term “oil” is understood to mean a nonaqueous fatty substance which is liquid at ambient temperature (25° C.) and atmospheric pressure (760 mmHg).

The oil can be chosen from volatile oils and/or nonvolatile oils, and their mixtures.

The oil or oils can be present in the composition according to the invention in a content ranging for example from 0.1% to 30% by weight, preferably from 1% to 20% by weight, with respect to the total weight of the composition.

The term “volatile oil” is understood to mean, within the meaning of the invention, an oil capable of evaporating on contact with the skin or with the keratinous fibre in less than one hour at ambient temperature and atmospheric pressure. The volatile organic solvent or solvents and the volatile oils of the invention are volatile cosmetic organic solvents and oils which are liquid at ambient temperature and which have a nonzero vapour pressure, at ambient temperature and atmospheric pressure, ranging in particular from 0.13 Pa to 40 000 Pa (10⁻³ to 300 mmHg), in particular ranging for example from 1.3 Pa to 13 000 Pa (0.01 to 100 mmHg) and more particularly ranging for example from 1.3 Pa to 1300 Pa (0.01 to 10 mmHg).

The term “nonvolatile oil” is understood to mean an oil which remains on the skin or the keratinous fibre at ambient temperature and atmospheric pressure for at least several hours and which has in particular a vapour pressure of less than 10⁻³ mmHg (0.13 Pa). These oils can be hydrocarbon oils, silicone oils, fluorinated oils or their mixtures.

The term “hydrocarbon oil” is understood to mean an oil comprising mainly hydrogen and carbon atoms and optionally oxygen, nitrogen, sulphur and phosphorus atoms. Volatile hydrocarbon oils can be chosen from hydrocarbon oils having from 8 to 16 carbon atoms, in particular branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, for example the oils sold under the Isopar or Permethyl tradenames, branched C₈-C₁₆ esters, isohexyl neopentanoate, and their mixtures. Other volatile hydrocarbon oils, such as petroleum distillates, in particular those sold under the Shell Solt name by Shell, can also be used. Preferably, the volatile solvent is chosen from volatile hydrocarbon oils having from 8 to 16 carbon atoms and their mixtures.

Use may also be made, as volatile oils, of volatile silicones, such as, for example, volatile linear or cyclic silicone oils, in particular those having a viscosity ≦8 centistokes (8×10⁻⁶ m²/s) and having in particular from 2 to 7 silicon atoms, these silicones obviously comprising alkyl or alkoxy groups having from 1 to 10 carbon atoms. Mention may in particular be made, as volatile silicone oil which can be used in the invention, of octamethylcyclotetrasiloxane, decamethylcyclopentasiloxane, dodecamethylcyclohexa-siloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may also be made of the volatile linear alkyltrisiloxane oils of general formula (I) 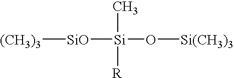
where R represents an alkyl group comprising from 2 to 4 carbon atoms, one or more hydrogen atoms of which can be substituted by a fluorine or chlorine atom.

Mention may be made, among the oils of general formula (I), of:

3-butyl-1,1,1,3,5,5,5-heptamethyltrisiloxane,

3-propyl-1,1,1,3,5,5,5-heptamethyltrisiloxane, and

3-ethyl-1,1,1,3,5,5,5-heptamethyltrisiloxane,

corresponding to the oils of formula (I) for which R is respectively a butyl group, a propyl group or an ethyl group.

Use may also be made of volatile fluorinated solvents, such as nonafluoromethoxybutane or perfluoromethylcyclopentane.

The composition can also comprise at least one nonvolatile oil, chosen in particular from nonvolatile hydrocarbon oils and/or silicone oils and/or fluorinated oils.

Mention may in particular be made, as nonvolatile hydrocarbon oil, of:

hydrocarbon oils of vegetable origin, such as triesters of fatty acids and of glycerol, the fatty acids of which can have varied chain lengths from C₄ to C₂₄, it being possible for these chains to be linear or branched and saturated or unsaturated; these oils are in particular wheat germ, sunflower, grape seed, sesame, maize, apricot kernel, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; or even triglycerides of caprylic/capric acids, such as those sold by Stéarineries Dubois or those sold under the names Miglyol 810, 812 and 818 by Dynamit Nobel,

synthetic ethers having from 10 to 40 carbon atoms;

linear or branched hydrocarbons of mineral or synthetic origin, such as liquid petrolatum, polydecenes, hydrogenated polyisobutene, such as Parleam oil, squalane and their mixtures;

synthetic esters, such as the oils of formula R₁COOR₂ in which R₁ represents the residue of a linear or branched fatty acid comprising from 1 to 40 carbon atoms and R₂ represents a hydrocarbon chain, in particular a branched hydrocarbon chain, comprising from 1 to 40 carbon atoms, provided that R₁+R₂ is ≧10, such as, for example, Purcellin oil (ketostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, octanoates, decanoates or ricinoleates of alcohols or of polyalcohols, such as propylene glycol dioctanoate; hydroxylated esters, such as isostearyl lactate or diisostearyl malate; and pentaerythritol esters;

fatty alcohols comprising a branched and/or unsaturated carbon chain having from 12 to 26 carbon atoms which are liquid at ambient temperature, such as octyldodecanol, isostearyl alcohol, oleyl alcohol, 2-hexyldecanol, 2-butyloctanol or 2-undecylpentadecanol;

higher fatty acids, such as oleic acid, linoleic acid or linolenic acid;

carbonates;

acetals;

citrates;

and their mixtures.

The nonvolatile silicone oils which can be used in the composition according to the invention include polydimethylsiloxanes (PDMSs) which are nonvolatile, polydimethylsiloxanes comprising pendent alkyl or alkoxy groups and/or alkyl or alkoxy groups at the end of the silicone chain, groups each having from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethyl-siloxysilicates.

The fluorinated oils which can be used in the invention are in particular fluorosilicone oils, fluorinated polyethers or fluorinated silicones, such as disclosed in the document EP-A-847 752.

Silicone Agent

The composition according to the invention advantageously comprises a silicone agent which can be an oxyalkylenated silicone.

The oxyalkylenated silicones are preferably chosen from the compounds of general formula (I), also known as dimethicone copolyols: 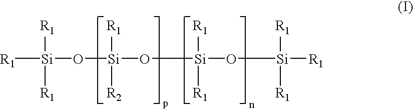
in which formula:

R₁, which are identical or different, represent a hydrogen atom, a linear or branched C₁-C₃₀ alkyl radical or phenyl radical,

R₂, which are identical or different, represent —(CₓH₂ₓ)—(OC₂H₄)ₐ—(OC₃H₆)_{b}—OR₃,

R₃, which are identical or different, are chosen from a hydrogen atom, a linear or branched alkyl radical having from 1 to 12 carbon atoms or a linear or branched acyl radical having from 2 to 12 carbon atoms,

n varies from 0 to 1000,

p varies from 1 to 30,

a varies from 0 to 50,

b varies from 0 to 50,

a+b is greater than or equal to 1,

x varies from 1 to 5,

the number-average molecular weight being greater than or equal to 15 000 and preferably between 25 000 and 75 000.

Use is preferentially made of the oxyalkylenated silicones of general formula (I) which correspond to at least one of and preferably all the following conditions:

R₁ denotes the methyl radical,

R₃ represents a hydrogen atom, a methyl radical or an acetyl radical and preferably hydrogen,

p varies from 8 to 20,

a is between 5 and 40 and preferably between 15 and 30,

b is between 5 and 40 and preferably between 15 and 30,

x is equal to 2 or 3,

n varies from 20 to 600, preferably from 50 to 500 and more particularly so from 100 to 300. Such silicones are, for example, described in U.S. Pat. No. 4,311,695, which is included by way of reference.

Dimethicone copolyols have in particular been presented by Dow Corning during the 17th International Congress of the IFSCC of October 1992 and reported in the paper “Water-soluble dimethicone copolyol waxes for personal care industry” by Linda Madore et al., pages 1 to 3.

These dimethicone copolyols are polydimethylsiloxanes (PDMSs) comprising one or more ether functional groups which are soluble in water (oxyalkylene, in particular oxyethylene and/or oxypropylene).

Such dimethicone copolyols are sold in particular by Goldschmidt under the name Abil B8851 or ABIL B88183. Mention may also be made of the compounds KF 351 to 354 and KF 615 A sold by Shin Etsu or DMC 6038 from Wacker.

The derivatives of dimethicone copolyols which can be used in the invention include in particular dimethicone copolyols possessing a phosphate, sulphate, myristamidopropyidimethylammonium chloride, stearate, amine or glycomodified group, and the like. Use may be made, as derivatives of dimethicone copolyols, in particular of the compounds sold by Siltech under the name Silphos A100, Siltech Amine 65, Silwax WDIS or Myristamido Silicone Quat or by Phoenix under the name Pecosil PS 100.

Use may also be made of the derivatives sold by Wacker under the name VP 1661 or by Dow Corning under the name 2501 Cosmetic Wax.

The silicones which are the most particularly preferred are, for example, those sold by Dow Corning under the trade name Q2-5220 and by Rhone Poulenc under the name Mirasil DMCO.

The silicone agent can represent from 0.01 to 5% by weight, with respect to the total weight of the composition, preferably from 0.1 to 2% by weight.

Colouring Material

The composition according to the invention can also comprise at least one colouring material, such as pulverulent materials, fat-soluble dyes or water-soluble dyes.

The pulverulent colouring materials can be chosen from pigments and pearlescent agents.

The pigments can be white or coloured, inorganic and/or organic and coated or noncoated. Mention may be made, among inorganic pigments, of titanium dioxide, optionally treated at the surface, zirconium, zinc or cerium oxides, and also iron or chromium oxides, manganese violet, ultramarine blue, chromium hydrate and ferric blue. Mention may be made, among organic pigments, of carbon black, pigments of D & C type and lakes, based on cochineal carmine, of barium, strontium, calcium or aluminium.

The pearlescent agents can be chosen for example from white pearlescent pigments, such as mica covered with titanium oxide or with bismuth oxychloride, coloured pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride. The fat-soluble dyes include, for example, Sudan red, D&C Red 17, D&C Green 6, β-carotene, soybean oil, Sudan brown, D&C Yellow 11, D&C Violet 2, D&C Orange 5, quinoline yellow or annatto. These colouring materials can be present in a content ranging for example from 0.01 to 30% by weight, with respect to the total weight of the composition.

The composition of the invention can additionally comprise any additive, including those conventionally used in cosmetics, such as antioxidants, preservatives, fibres, fragrances, neutralizing agents, gelling agents, thickeners, vitamins, coalescence agents, plasticizers and their mixtures.

Fibres

The composition according to the invention can additionally comprise fibres which make possible an improvement in the lengthening effect. The term “fibre” should be understood as meaning an object with a length L and a diameter D such that L is much greater than D, D being the diameter of the circle in which the cross section of the fibre is framed. In particular, the L/D ratio (or aspect ratio) is chosen within the range from 3.5 to 2500, in particular from 5 to 500 and more particularly from 5 to 150.

The fibres which can be used in the composition of the invention include fibres of synthetic or natural and inorganic or organic origin. They can be short or long, individual or organized, for example plaited, and hollow or solid. They can have any shape and can in particular be circular or polygonal (square, hexagonal or octagonal) in cross section, according to the specific application envisaged. In particular, their ends are blunted and/or polished to prevent injury. In particular, the fibres have a preferred length ranging for example from 1 μm to 10 mm, in particular from 0.1 mm to 5 mm and more particularly from 0.3 mm to 3.5 mm. Their cross section can be included within a circle with a diameter ranging for example from 2 nm to 500 μm, in particular ranging for example from 100 nm to 100 μm and more particularly ranging for example from 1 μm to 50 μm. The weight or count of the fibres is often given in denier or decitex and represents the weight in grams per 9 km of yarn. The fibres according to the invention can in particular have a count chosen within the range from 0.15 to 30 denier and in particular from 0.18 to 18 denier. The fibres which can be used in the composition of the invention include those chosen from rigid and nonrigid fibres. They can be of synthetic or natural and inorganic or organic origin. Furthermore, the fibres may or may not be surface treated, may or may not be coated and may or may not be coloured. Mention may be made, as examples of fibres which can be used in the composition according to the invention, of fibres which are not rigid, such as polyamide (Nylon®) fibres, or fibres which are rigid, such as polyimideamide fibres, for example those sold under the Kermel® or Kermel Tech® names by Rhodia, or poly(p-phenylene terephthalamide) (or aramid) fibres, sold in particular under the Kevlar® name by DuPont de Nemours.

The fibres can be present in the composition according to the invention in a content ranging for example from 0.01% to 10% by weight, with respect to the total weight of the composition, in particular from 0.1% to 5% by weight and more particularly from 0.3% to 3% by weight.

Cosmetic Active Principles

Mention may in particular be made, as examples of cosmetic active principles which can be used in the compositions according to the invention, of antioxidants, preservatives, fragrances, neutralizing agents, emollients, moisturizing agents, vitamins and screening agents, in particular sunscreens.

Of course, a person skilled in the art will take care to choose the optional additional additives and/or their amounts so that the advantageous properties of the composition according to the invention are not, or not substantially, detrimentally affected by the addition.

The composition according to the invention can be packaged in a container delimiting at least one compartment which comprises the composition, the container being closed by a closure part.

The container is preferably used in combination with an applicator, in particular in the form of a brush comprising an arrangement of hairs held by a twisted wire. Such a twisted brush is described in particular in U.S. Pat. No. 4,887,622. It can also be in the form of a comb comprising a plurality of applicational parts, obtained in particular from moulding. Such combs are described, for example, in Patent FR 2 796 529. The applicator can be integral with the container, such as described, for example, in Patent FR 2 761 959. Advantageously, the applicator is integral with a rod which, itself, is integral with the closure part.

The closure part can be coupled to the container by screwing. Alternatively, the closure part and the container can be coupled other than by screwing, in particular via a bayonet mechanism, by latching or by clamping. The term “latching” is understood to mean in particular any system which involves surmounting a flange or ring of material by elastic deformation of a portion, in particular of the closure part, and then by returning to the elastically unstressed position of the portion after the flange or ring has been surmounted.

The container can be at least partly made of thermoplastic. Mention may be made, as examples of thermoplastics, of polypropylene or polyethylene. Alternatively, the container is made of nonthermoplastic material, in particular of glass or of metal (or alloy).

The container is preferably equipped with a wringer positioned in the vicinity of the opening of the container. Such a wringer makes it possible to wipe the applicator and optionally the rod to which it may be integrally attached. Such a wringer is described, for example, in Patent FR 2 792 618.

The examples which follow are presented by way of illustration and without implied limitation of the invention. Unless otherwise indicated, the amounts are given in grams.

### EXAMPLE 1

The mascara with the following composition is prepared:

| | |
|---|---|
| Rice bran wax | 5.6 |
| Candelilla wax | 2 |
| Beeswax | 5.2 |
| Carnauba wax | 6 |
| (Hydroxystearoyl)stearate of C₁₈-C₃₈ fatty alcohols | 4.1 |
| (Kester K82 P from Koster Keunen) | |
| Oxypropylenated (20 PO)/oxyethylenated (20 EO) | 0.5 |
| polydimethylsiloxane (DC Q2-5220 Resin Modifier | |
| from Corning) | |
| Gum arabic | 2.5 |
| Hydroxyethyl cellulose | 0.5 |
| Styrene/acrylates/ammonium methacrylate copolymer | 4 (AM*) |
| comprising 40% of AM in water with butylene glycol | |
| and sodium laureth-12 sulphate (Syntran 5760) | |
| Simethicone | 0.15 |
| Polyethylene beads, 6-14 μm (Micropoly 200L from | 0.5 |
| Micropowders) | |
| Amorphous silica microspheres, 5 μm (Sunsphere H51 | 1 |
| from Asahi Glass) | |
| PEG-40 stearate (Myrj 52P from Unichema) | 1.5 |
| Triethanolamine | 2.4 |
| Stearic acid | 4.6 |
| D-Panthenol | 0.5 |
| Di(tert-butyl)-4-hydroxytoluene | 0.1 |
| Black iron oxide | 7 |
| Preservatives | q.s. |
| Water | q.s. for 100 |

*AM: active materials

This mascara exhibits a viscosity, measured according to the method indicated above, of 10 Pa·s. It is easy to apply to the eyelashes and forms a smooth and homogeneous volumizing deposited layer exhibiting good hold over time.

The above written description of the invention provides a manner and process of making and using it such that any person skilled in this art is enabled to make and use the same, this enablement being provided in particular for the subject matter of the appended claims, which make up a part of the original description and including a composition for coating keratinous fibres comprising an aqueous phase, at least one filler, where the filler represents at least 0.1% by weight with respect to the total weight of the composition, at least one film-forming polymer in the form of solid particles dispersed in the aqueous phase and at least one wax such that the total content of wax(es) represents at least 21% by weight with respect to the total weight of the composition, the composition exhibiting a viscosity at 25° C. of less than or equal to 13.5 Pa·s.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition exhibiting a viscosity at 25° C. of less than or equal to 13.5 Pa·s and comprising an aqueous phase, at least one filler, at least one film-forming polymer in the form of solid particles dispersed in the aqueous phase, and at least one wax,
wherein the filler is present in an amount of at least 0.1% by weight with respect to the total weight of the composition and the total content of wax(es) is at least 21% by weight with respect to the total weight of the composition, the composition.

2. The composition according to claim 1, wherein said compositon has a viscosity of 8 to 13.5 Pa·s.

3. The composition according to claim 1, wherein the wax is present in a content of greater than or equal to 22% by weight, with respect to the total weight of the composition.

4. The composition according to claim 1, wherein it comprises at least one wax having a tack greater than or equal to 0.1 N.s and a hardness of less than or equal to 3.5 MPa.

5. The composition according to claim 4, comprising at least one wax that is a C₂₀-C₄₀ alkyl(hydroxystearyloxy)stearate.

6. The composition according to claim 1, wherein the filler represents at least 0.5% by weight with respect to the total weight of the composition.

7. The composition according to claim 1, wherein the filler represents from 0.1 to 25% by weight with respect to the total weight of the composition.

8. The composition according to claim 1, comprising at least one inorganic filler and at least one organic filler.

9. The composition according to claim 1, wherein the inorganic filler is chosen from talc, mica, silica, kaolin, starch, hydroxyapatite, boron nitride, hollow silica microspheres, glass or ceramic microcapsules, and mixtures thereof.

10. The composition according to claim 1, comprising at least one organic filler selected from the group consisting of powders formed of polyamide, of poly-β-alanine or of polyethylene, lauroyllysine, starch, powders formed of tetrafluoroethylene polymers, expanded polymer hollow microspheres, acrylic powders, acrylate copolymers, poly(methyl methacrylate) (PMMA), 12-hydroxystearic acid oligomer stearate, silicone resin microbeads, precipitated calcium carbonate, magnesium carbonate, basic magnesium carbonate, metal soaps derived from organic carboxylic acids having from 8 to 22 carbon atoms, heat-expandable particles, and their mixtures.

11. The composition according to claim 1, wherein it comprises at least one inorganic filler chosen from silicas and at least one organic filler chosen from polyethylene powders.

12. The composition according to claim 8, wherein the inorganic filler is present in a proportion of 0.1 to 10% by weight with respect to the total weight of the composition, and the organic filler is present in a proportion of 0.05 to 5% by weight with respect to the total weight of the composition.

13. The composition according to claim 1, wherein the aqueous phase is present in an amount of 3% to 80% by weight with respect to the total weight of the composition.

14. The composition according to claim 1, wherein it comprises at least one film-forming polymer in a content of dry matter of 0.1% to 30% by weight with respect to the total weight of the composition.

15. The composition according to claim 14, wherein the film-forming polymer is present in an amount of 0.5% to 20% by weight.

16. The composition according to claim 14, wherein the composition comprises at least 2% by weight of dry matter of film-forming polymer.

17. The composition according to claim 14, comprising an acrylic film-forming polymer in the form of solid particles in dispersion in the aqueous phase.

18. The composition according to claim 17, wherein it comprises at least one film-forming acrylic polymer in the form of solid particles in dispersion in the aqueous phase, the polymer resulting from the polymerization of at least one monomer possessing ethylenic unsaturation chosen from α,β-ethylenic carboxylic acids, their esters and their amides.

19. The composition according to claim 18, wherein the α,β-ethylenic unsaturated carboxylic acid is chosen from acrylic acid, methacrylic acid, crotonic acid, maleic acid, itaconic acid and their mixtures.

20. The composition according to claim 18, the polymer resulting from the polymerization of at least one monomer possessing ethylenic unsaturation chosen from α,β-ethylenic carboxylic esters wherein the ester is chosen from C₁-C₃₀ alkyl(meth)acrylates, aryl(meth)acrylates, and hydroxyalkyl(meth)acrylates.

21. The composition according to claim 18, the polymer resulting from the polymerization of at least one monomer possessing ethylenic unsaturation chosen from α,β-ethylenic carboxylic amides chosen from N-((C₂-C₁₂)alkyl)(meth)acrylamides.

22. The composition according to claim 18, wherein the acrylic polymer comprises at least one styrene monomer.

23. The composition according to claim 1, wherein said composition is a mascara.

24. A process for making up keratinous fibres, wherein the composition defined in claim 1 is applied to keratinous fibres.
